(19) United States
(12) Patent Application Publication
(10) Pub. No.: US 2023/0414491 A1
(43) Pub. Date: Dec. 28, 2023

(54) KITS, SYSTEMS, AND METHODS FOR REDUCING SURGICAL SITE INFECTIONS

(71) Applicant: Nanovis, LLC, Columbia City, IN (US)

(72) Inventors: David Alan DETWILER, Columbia City, IN (US); Matthew HEDRICK, Carmel, IN (US)

(73) Assignee: Nanovis, LLC, Columbia City, IN (US)

(21) Appl. No.: 18/334,155

(22) Filed: Jun. 13, 2023

Related U.S. Application Data
(63) Continuation of application No. PCT/US2021/063066, filed on Dec. 13, 2021.
(60) Provisional application No. 63/125,135, filed on Dec. 14, 2020.

(51) Int. Cl. A61K 9/00; A61K 45/06

(52) U.S. Cl. CPC A61K 9/0024; A61K 45/06

(57) ABSTRACT

The present disclosure relates to a kit for preventing, reducing, and/or treating surgical site infections, the kit comprising two or more antimicrobial components directed to two or more different post-surgical tissue depths in a subject. The present disclosure further relates to an antimicrobial surgical system, the system comprising two or more antimicrobial components directed to two or more different post-surgical tissue depths in a subject for preventing, reducing, and/or treating surgical site infections. The present disclosure further relates to a method of preventing, reducing, and/or treating one or more surgical site infections, the method comprising: providing an antimicrobial surgical system comprising: two or more antimicrobial components directed to two or more different post-surgical tissue depths in a subject; and applying said antimicrobial surgical system to a subject under conditions effective to prevent, reduce, and/or treat one or more surgical site infections. Also, disclosed is the method of assembling the kit.

This application is a Bypass Continuation Application to PCT International Application No. PCT/US2021/063066, filed Dec. 13, 2021, and entitled “Kits, Systems, and Methods For Reducing Surgical Site Infections”, which claims priority benefit under 35 U.S.C. § 119(e) of U.S. Provisional Patent Application Ser. No. 63/125,135, filed Dec. 14, 2020. Both applications and the disclosures of each are hereby incorporated herein by reference in their entirety.

### TECHNICAL FIELD

The present disclosure relates to kits, systems, and methods for reducing surgical site and post-operative infections.

### BACKGROUND OF THE INVENTION

Existing procedures for post-operation recovery are limited and present risk of infection. Broad spectrum antibiotics are frequently administered to a subject recovering from surgery in a prophylactic manner, often resulting in undesirable side effects that could be avoided and could further complicate recovery from surgery. Surgical Site Infections (SSI)s involving implants are a serious and common issue, often resulting in detrimental results and reduced rates of recovery following surgery. There are added costs for patients in money and quality of life and additional costs on hospital systems. Surgical site infections can be overt and exist between 1% for a simple laminectomy to upwards of 10% for complex instrumented cases of spinal surgery (Peng et al., “Risk Factors for Surgical Site Infection After Spinal Surgery: A Systematic Review and Meta-Analysis Based on Twenty-Seven Studies,” World Neurosurg. 123:e318-329 (2019)). Spinal surgery with hardware has an infection rate of 1-4% (Parikh and Antony, “A Comprehensive Review of the Diagnosis and Management of Prosthetic Joint Infections in the Absence of Positive Cultures,” J. Infect. Publ. Health 9:545-556 (2016)). Occult infections, those that have no outward clinical signs, have been measured in revision surgeries to be between 9.3% and 40.2% (Hu and Lieberman, “Revision Spine Surgery in Patients Without Clinical Signs of Infection: How Often Are There Occult Infections in Removed Hardware?,” European Spine Journal 27:2491-95 (2018)). Occult infections may cause delays in healing, lead to loosening of hardware or failure to fuse, creating a pseudoarthrosis. The skin is the primary site of infection and additional cleaning steps of the skin has been shown to reduce post-surgical skin infections (Morrison et al., “Single vs Repeat Surgical Skin Preparations for Reducing Surgical Site Infection After Total Joint Arthroplasty: A Prospective, Randomized, Double-Blinded Study,” The Journal of Arthroplasty 31:1289-1294 (2016)). However, this study did not reduce deep infections.

There remains a need to improve methods of recovery after surgical procedures. In particular, there is a need to prevent, ameliorate, and treat infections following surgery.

### SUMMARY OF THE INVENTION

A first aspect of the present disclosure relates to a kit for preventing, reducing, and/or treating surgical site infections. The kit comprises two or more antimicrobial components directed to two or more different post-surgical tissue depths in a subject.

A second aspect of the present disclosure relates to a method of assembling the kit described herein.

A third aspect of the present disclosure relates to an antimicrobial surgical system. The system comprises two or more antimicrobial components directed to two or more different post-surgical tissue depths in a subject for preventing, reducing, and/or treating surgical site infections.

A fourth aspect of the present disclosure relates to a method of assembling the system described herein.

A fifth aspect of the present disclosure relates to a method of preventing, reducing, and/or treating one or more surgical site infections. The method includes providing an antimicrobial surgical system including: two or more antimicrobial components directed to two or more different post-surgical tissue depths in a subject; and applying said antimicrobial surgical system to a subject under conditions effective to prevent, reduce, and/or treat one or more surgical site infections.

### BRIEF DESCRIPTION OF THE DRAWINGS

The accompanying drawings, which are incorporated in and constitute a part of the specification, illustrate embodiments of the inventions and together with the detailed description herein, serve to explain the principles of the inventions. It is emphasized that, in accordance with the standard practice in the industry, various features may or may not be drawn to scale. In fact, the dimensions of the various features may be arbitrarily increased or reduced for clarity of discussion. The drawings are only for purposes of illustrating embodiments of inventions of the disclosure and are not to be construed as limiting the inventions.

FIG. 1 shows a four-hour bacterial adhesion plate counts of S. aureus in various surgical hardware, including for example, antimicrobial surgical hardware. Column 1 (Control Ti) represents control titanium alloy ELI Grade 23; Column 2 (Anodized) represents control titanium that has been anodized for 10 minutes at 20 volts with no further surface modification; Column 3 (A+CaP) represents the anodized surface from column 2 with the addition of a thin amorphous calcium phosphate layer 3-9 nm in thickness; Column 4 (A+CaP Stemulate) represents the column 3 sample with the adhesion of a Cook Biotech human platelet lysate consisting of active enzymes, growth factors, and other proteins; Column 5 (A+CaP FBS) represents the sample from column 3 with the addition of Gibco fetal bovine serum; and Column 6 (A+Thick CaP) represents the surface from column 2 with the addition of a thick amorphous calcium phosphate layer 30-50 nm in thickness. Bacteria cultures of 10\hat{ }6 CFU/mL Streptococcus aureus were allowed to adhere to the surface for 4 hours. Bacteria were recovered from the surface with an ultrasonic wash in media that contained a surfactant to aid in cell recovery. Ultrasonicated samples were plated and counted after overnight culture at 37° C. Sample counts were normalized to the control titanium surface for reference;

FIG. 2A shows the two layer antimicrobial nanotube structure created by subsequent anodization steps utilizing hydrofluoric acid (HF) in the first step to create the top layer, followed by the second anodization step utilizing ammonium fluoride with 3% water in 97% ethylene glycol (EG) solution. The first layer is required to be at least 100 nm and the second layer is also required to be at least 100 nm. It is extremely difficult if not impossible to create nanotubes of 70 nm diameter that are longer than about 150 nm created in an HF aqueous solution. The surface morphology created by the HF anodization has been demonstrated to the FDA to increase the mineralization of extracellular matrix in vitro. The EG anodization process can provide additional length to the nanotubes without altering the surface morphology that the cells interact with to create the biological response. The increased surface area created with EG anodization allows for the increased loading of nanotubes with antimicrobials onto the surface or into the volume created by the nanotube;

FIG. 2B shows the in vitro biocompatibility in a 24- and 72-hour cell viability assay using human osteoblasts. The dye used is a tetrazolium dye MTT dimethylthiazol-2-yl)-2,5-diphenyltetrazolium bromide (MTT) that is converted to a brown product that can be quantified. The conversion uses an NAD(P)H-dependent cellular oxidoreductase enzyme to measure viable cells. The measurements were compared to control titanium and anodized titanium with thick calcium phosphate without antimicrobials;

FIG. 3A shows 1-Layer AM surface bacterial inhibition zones out to 21 Days. Cultures of Staphylococcus aureus and Escherichia coli were spread on plates and titanium discs with the antimicrobial surface eluted to various time points were placed on the spread bacterial cultures. Culturing overnight allowed the colonies to grow. Areas around the antimicrobial disks that limited growth of the bacterial colonies are referred to as inhibition zones. Inhibition zones were demonstrated out to at least 21 days indicating extended antimicrobial properties of the surface;

FIG. 3B shows 1-Layer AM surface with chlorhexidine bacterial inhibition zones on Day 1. Chlorhexidine was added to the surface to create a surface with more than one antimicrobial component to address possible antimicrobial resistance issues. The chlorhexidine provides a short term burst of antimicrobial capability to the antimicrobial surface;

FIG. 4 shows extraction torque values from a horse pin external fixator study;

FIG. 5 shows the horse pin histology after 5-week external fixator study. Increased bone implant contact on nanosurfaced and antimicrobial nanosurfaced pins relative to control titanium;

FIG. 6 shows gelatin foams with varying levels of Rifampicin and Minocycline antibiotics loaded into the gels following freezing and lyophilization; and

FIG. 7 shows a wound insert gelatin foam bubble structure after freezing and lyophilization to measure wall thickness.

### DETAILED DESCRIPTION OF THE INVENTION

A first aspect relates to a kit for preventing, reducing, and/or treating surgical site infections. The kit comprises two or more antimicrobial components directed to two or more different post-surgical tissue depths in a subject.

It is to be appreciated that certain aspects, modes, embodiments, variations, and features of the present disclosure are described below in various levels of detail in order to provide a substantial understanding of the present technology. The definitions of certain terms as used in this specification are provided below. Unless defined otherwise, all technical and scientific terms used herein generally have the same meaning as commonly understood by one of ordinary skill in the art to which this disclosure belongs.

As used herein, the term “about” means that the numerical value is approximate and small variations would not significantly affect the practice of the disclosed embodiments. Where a numerical limitation is used, unless indicated otherwise by the context, “about” means the numerical value can vary by ±1 or ±10%, or any point therein, and remain within the scope of the disclosed embodiments.

Where a range of values is described, it should be understood that intervening values, unless the context clearly dictates otherwise, between the upper and lower limit of that range and any other stated or intervening value in other stated ranges, may be used in the embodiments described herein.

The term “cell or group of cells” is intended to encompass single cells as well as multiple cells either in suspension, monolayers, or biofilms. Whole tissues also constitute a group of cells.

As used herein, the terms “subject”, “individual”, or “patient,” are used interchangeably, and mean any animal, including mammals, such as mice, rats, other rodents, rabbits, dogs, cats, swine, cattle, sheep, horses, swine, goat, deer, elk, or primates, such as humans. Preferably, the subject is human.

For purposes of this and other aspects of the disclosure, the target “subject” or “patient” encompasses any vertebrate, such as an animal, preferably a mammal, more preferably a human. In the context of administering a kit, system, or method of the disclosure for purposes of reducing, ameliorating, and/or preventing a surgical site infection, the target subject encompasses any subject that has or is at risk of developing a surgical site infection. Particularly susceptible subjects include adults and elderly adults. However, any infant, juvenile, adult, or elderly adult that has or is at risk of having a surgical site infection can be treated in accordance with the kits, systems, and methods of the present disclosure. In one embodiment, the subject is an infant, a juvenile, or an adult.

It is further appreciated that certain features described herein, which are, for clarity, described in the context of separate embodiments, can also be provided in combination in a single embodiment. Conversely, various features which are, for brevity, described in the context of a single embodiment, can also be provided separately or in any suitable sub-combination.

The kits described in the present aspect may be used to prevent, reduce, and/or treat one or more surgical site infections.

Preventing, reducing, and treating one or more surgical site infections as described herein includes therapeutic treatment and prophylactic or preventative measures, where the objective is to prevent, slow down, alleviate, and/or lessen the infection (e.g., surgical site infection or infection at or near a post-surgical site) or its complications, preventing or inhibiting it from manifesting, preventing, or inhibiting it from recurring, merely preventing or inhibiting it from worsening, curing the disease, reversing the progression of the infection, prolonging a patient's life or life expectancy, ameliorating the infection, or a therapeutic effort to effect any of the aforementioned, even if such therapeutic effort is ultimately unsuccessful.

As used herein, the phrase “therapeutically effective amount” means an amount of an antimicrobial agent that elicits the biological or medicinal response that is being sought in a tissue, system, animal, individual or human by a researcher, veterinarian, medical doctor, or other clinician. As such, the therapeutic effect can be a decrease in the severity of symptoms associated with the disorder and/or inhibition (partial or complete) of progression of the disorder, or improved treatment, healing, prevention or elimination of a disorder, or side-effects. The amount needed to elicit the therapeutic response can be determined based on the age, health, size, and sex of the subject. Optimal amounts can also be determined based on monitoring of the subject's response to treatment. The term “treatment” or “treat” may include effective inhibition, suppression or cessation of post-surgical infection symptoms so as to prevent or delay the onset, retard the progression, or ameliorate the symptoms of a post-surgical infection.

One goal of treatment is the amelioration, either partial or complete, either temporary or permanent, of patient symptoms, including inflammation, physical manifestations of a surgical site infection, epithelial damage, and/or any early markers of post-surgical infection. The kits, systems, and methods described herein may be administered before onset of any symptoms or manifestation of an infection, for example, may be administered during a surgery or immediately following a surgery, or at a period of time following surgery but before any symptoms of surgical site infection appear. Any amelioration is considered successful treatment. This is especially true as amelioration of some magnitude may allow reduction of other medical or surgical treatment which may be more toxic or invasive to the patient. In one embodiment, the post-surgical infection and/or the condition resulting from post-surgical infection is prevented. In another embodiment, the post-surgical infection and/or the condition resulting from post-surgical infection is treated.

The kits, systems, and methods described herein includes two or more antimicrobial components, and the two or more antimicrobial components may, in one embodiment, be directed to two or more different post-surgical tissue depths in a subject.

In one embodiment, the two or more components are selected from: (i) a first component, said first component directed to a first post-surgical tissue depth comprising surgical hardware; (ii) a second component, said second component directed to a second post-surgical tissue depth comprising a deep tissue layer; (iii) a third component, said third component directed to a third post-surgical tissue depth comprising a superficial tissue layer; and/or (iv) a fourth component, directed to a fourth post-surgical tissue depth comprising skin.

Tissues as described herein include aggregates or groups of cells organized to perform one or more specific functions. Tissues as described herein are characterized as layers. There are four basic tissue types—epithelial tissue, connective tissue, muscle tissue, and nerve tissue. See Ross et al., “Tissues: Concept and Classification,” HISTOLOGY: A TEXT AND ATLAS (Fourth Ed.) (2003), which is hereby incorporated by reference in its entirety. Epithelial tissue covers body surfaces, lines body cavities, and forms glands. Connective tissue underlies or supports the other three basic tissues, both structurally and functionally. Muscle tissue is made up of contractile cells and is responsible for movement. Nerve tissue receives, transmits, and integrates information from outside and inside the body to control activities of the body. As described herein, a first post-surgical tissue depth comprising surgical hardware is generally deepest inside of a subject, relative to the subject's epithelial tissue (which is found in a fourth post-surgical tissue depth as described herein). Thus, a first post-surgical tissue depth (e.g., depth of one or more surgical hardware elements) is furthest from a fourth post-surgical tissue depth (e.g., depth of skin) as described herein.

A first post-surgical tissue depth comprising surgical hardware as described herein includes a depth inside tissue of a subject, whereby the surgical hardware is located. For example, following an implant surgery, the post-surgical tissue depth comprising surgical hardware is the depth of tissue whereby the implant is located.

A second post-surgical tissue depth comprising a deep tissue layer as described herein includes a depth inside tissue of a subject whereby deep tissue is located. Deep tissue as described herein includes a layer of tissue directly above, surrounding, and/or adjacent to the tissue depth of one or more surgical hardware elements. For example, following an implant surgery, the post-surgical tissue depth comprising a deep tissue layer includes the layer of tissue directly above, surrounding, and/or adjacent to the location of the implant. In one embodiment, such deep tissue may include spinal tissue, connective tissue, muscular tissue, and may further include inner layers of muscles, fascia, connective tissues, and any combination thereof.

A third post-surgical tissue depth comprising a superficial tissue layer as described herein includes a depth inside tissue of a subject, whereby superficial tissue is located. Superficial tissue as described herein includes a layer of tissue between a deep tissue layer and an epithelial tissue layer. For example, following an implant surgery, the post-surgical tissue depth comprising a superficial tissue layer includes layers of tissue between a deep tissue layer and a dermal or skin layer, in particular the dermal or skin layer comprising the entry point of a surgical procedure. In one embodiment, the superficial tissue layer includes a thin layer of loose fatty connective tissue underlying the dermis and binding it to the parts beneath. The superficial tissue layer may include, for example, fascia, hypodermis, and tela subcutanea.

Fascia as described herein includes connective tissue that surrounds muscles, groups of muscles, blood vessels, and nerves, and the binding of those structures together. It consists of several layers: a superficial fascia (e.g., a superficial tissue layer as described herein), a deep fascia (e.g., a deep tissue layer as described herein), and a subserous of visceral fascia (e.g., a deep tissue layer as described herein). Superficial tissue may include a fascial sheet lying directly beneath the skin (e.g., a subcutaneous fat layer). Deep fascia comprises an intricate series of connective sheets and bands that hold the muscles and other structures in place throughout the body.

A fourth post-surgical tissue depth comprising skin as described herein includes a depth inside tissue of a subject, whereby skin is located. For example, following an implant surgery, the post-surgical tissue depth comprising a skin layer (also referred to herein as a dermal layer or external layer) includes outer layers of tissue, such as an incision site following an implant surgery. The tissue depth comprising the skin, includes components that rest on the skin and components that are in the skin. For example, a wound dressing, cover, or gel applied to a surface of a wound but not inside the wound is contemplated as being within the fourth post-surgical tissue depth. Likewise, a suture that would contact tissue both on top of the skin and within the skin are contemplated as being within the fourth post-surgical tissue depth.

When used together, a kit comprising two or more antimicrobial components, and the two or more antimicrobial components may, in one embodiment, be directed to two or more different post-surgical tissue depths in a subject, may prevent or reduce the bacteria in or on a wound or on an implant thereby preventing, reducing, or treating infection. The use of the components may reduce infections at various depths of a surgical wound or migration of bacteria from one depth of a surgical wound to another.

The kit, systems, and methods described herein may include an antimicrobial attachment surface and/or an antimicrobial agent.

An antimicrobial agent as described herein may include any agent or surface texture or property that when applied to a component prevents, stops, ameliorates, slows down microbial activity, for example, by imparting antimicrobial activity, to that component.

A microbe may be any organism the presence or growth or proliferation of which may be undesirable. For example, a microbe may be a bacteria strain, such as infectious bacteria. A bacteria strain may be gram-positive or gram-negative. Examples of bacteria include, but are not limited to, Staphylococcus aureus, Staphylococcus epidermidis, Escherichia coli, Acenitobacter baumanii, Klebsiella pneumoniae, and Pseudomonas aeruginosa. Other bacteria related or unrelated to any of the foregoing species and different strains thereof may also be included in the kit, system, and method as disclosed herein. It would be understood that a method of reducing a number of viable microbes hereby provided could be applied to numerous different microbes in addition to these examples. Some specific examples include strains of microbes known to be resistant or show low levels of susceptibility to treatment with known antibiotics. Examples include vancomycin-resistant Staphylococcus aureus, methicillin-resistant Staphylococcus aureus, a strain of multidrug-resistant Pseudomonas aeruginosa, or a strain of multidrug-resistant Escherichia coli. Some non-limiting examples of strains of bacteria that are known to be resistant or refractory to treatment with one or more antibiotics which are generally effective against other strains of said bacteria include: carbapenem-resistant Escherichia coli; Klebsiella pneumoniae; gentamicin, streptomycin and sulfonamide resistant Pseudomonas aeruginosa; and methicillin resistant Staphylococcus aureus which in some examples may also be resistant to erythromycin and tetracycline. Such strains show low or no susceptibility to exposure to various antibiotics that are otherwise bactericidal and/or bacteriostatic to other strains or other bacteria at comparable concentrations.

Other infectious or other microbes may also be included in the antimicrobial agents described herein. For example, a microbe may be a fungus, such as Candida albicans, Candida auris, or species of Aspergillis. Various antifungal compounds may be included in accordance with an aspect of the present disclosure. Non-limiting examples include clotrimazole, econazole, miconazole, terbinafine, fluconazole, ketoconazole, and amphotericin, or other compounds known to have antifungal activities. In some examples, combinations of any two or more of the foregoing antifungals or substances with antifungal activity may be administered concurrently in accordance with an aspect of the present disclosure. In some examples, any one or more of the foregoing antifungals or substances with antifungal activity may also be explicitly excluded from use in accordance with an aspect of the present disclosure. In some other examples, one or more of the foregoing antibiotics or substances with antibiotic activity may be used in combination with any one or more of the foregoing antifungals or substances with antifungal activity in accordance with an aspect of the present disclosure.

Antimicrobial as described herein includes inhibiting the growth, proliferation, viability, reproduction, infectivity, or number of microbes may be desirable. As used herein, reducing a number of viable microbes includes any of the foregoing effects on microbe colonies or populations. Included are bacteriostatic and bactericidal effects. An antimicrobial composition may be included in the kits, systems, and methods described herein with the result of inhibiting the growth, proliferation, viability, reproduction, infectivity, or number of microbes present, each and all of which are included in reducing a number of viable microbes. A reduction in a number of viable microbes may result from a strictly bactericidal effect, a bacteriostatic effect, or a combination of the two. Antimicrobial as described herein encompasses three subcategories: anti-colonization, anti-proliferation/bacteriostatic, and bactericidal. Anti-colonization surfaces may be drug based or structure/material based or a combination of these features. These surfaces may have the property of limiting bacterial attachment, proliferation, function, or biofilm formation on the surface. These surfaces may have the property of increasing eukaryotic cell attachment, proliferation, or function on a surface. This can be accomplished through nanostructures on metals or polymers that limit the contact of the stiff membranes of bacteria, effectively limiting the surface area that the bacteria can attach to on the surface.

A reduction in a number of viable microbes may be identified by any of a number of known methods. For example, a treatment may be applied to one of two otherwise identical samples, then the samples cultured to measure microbial growth following said treatment as compared to following absence of said treatment. If fewer microbes are present after culturing the sample to which said treatment had been applied relative to the untreated sample, the treatment reduced a number of viable microbes. A sample may be any surface, composition, liquid, substance, surface, tissue, or other material to which treatment as disclosed herein may be applied. In an example, applying the kits, systems, and/or methods described herein to a subject, such as a human or non-human animal subject, results in less infection (less in severity, less in duration, or both, or absence of infection) than results under similar circumstances, or than would have resulted, without treatment. For example, application of such treatment may slow growth of infectious microbes or otherwise render them more susceptible to a subject's immune system. Such examples of reduced infection are examples of reducing a number of viable microbes.

In some examples, a treatment as disclosed herein may slow or prevent proliferation of microbes and thereby hasten a reduction in number of viable microbes (e.g., increase susceptibility to a subject's immune system). In other examples, a treatment as disclosed herein may kill microbes without immediately eliminating or removing them. Both are examples of a treatment reducing a number of viable microbes.

In other examples, a reduction in a viable number of microbes might not result in a reduced duration, degree, or severity of an infection but may be evinced by culturing a sample and ascertaining an amount of microbial growth supported by such sample (following treatment as opposed to absent treatment). Other measures of a number of viable microbes may be used as well, such as quantitative measures of microbial markers (antigens, genetic material, etc.) present in a sample, or microscopic or other known detection method. In some examples, such reduction of a number of viable microbes may be evident within about 1 hr, about 2 hr, about 3 hr, about 4 hr, about 5 hr, about 6 hr, about 7 hr, about 8 hr, about 9 hr, about 10 hr, about 11 hr, about 12 hr, about 13 hr, about 14 hr, about 15 hr, about 16 hr, about 17 hr, about 18 hr, about 19 hr, about 20 hr, about 21 hr, about 22 hr, about 23 hr, about 24 hr, about 25 hr, about 26 hr, about 27 hr, about 28 hr, about 29, hr, about 30 hr, about 31 hr, about 32 hr, about 33 hr, about 34 hr, about 35 hr, about 36 hr, about 37 hr, about 38 hr, about 39 hr, about 40 hr, about 41 hr, about 42 hr, about 43 hr, about 44 hr, about 45 hr, about 46 hr, about 47 hr, about 48 hr, about 54 hr, about 60 hr, about 66 hr, about 72 hr, about 78 hr, about 84 hr, about 90 hr, about 96 hr, about 4.5 days, about 5 days, about 5.5 days, about 6 days, about 6.5 days, about 7 days, about 10 days, about 14 days, about 17 days, about 21 days, or about 28 days after administration of the kits, systems, and methods described herein. In this case, “about” includes within +/−15% of the duration indicated.

An antimicrobial composition may be applied to a surface, solution, or substance, as disclosed, in order to reduce a number of viable microbes on said surface or in such solution or substance, in accordance with the present disclosure. In an example, an antimicrobial may be applied or administered to a living subject such as a human. For example, an acute, chronic, sub-acute, sub-chronic, treatment-refractory, or other microbial infection, such as a bacterial or fungal infection, may be present in a subject such as a human subject. An antimicrobial composition may be applied or administered to such subject to reduce the number of viable microbes, such as to eliminate, remove, reduce, ameliorate, or otherwise treat such infection. In another example, such infection may be anticipated or a risk of such infection may be present, such as in an immunocompromised subject, or in conjunction with surgery or wound or trauma, or known or expected exposure to an infectious microbe, whereupon an antimicrobial composition may be administered prophylactically, to prevent the development of infection or proliferation of an infectious seed of microbe that may be present or suspected of being present. Such examples are included with reducing an amount of viable microbes as the term is used herein.

An antimicrobial composition may be administered by any of various medically known or accepted or approved means of applying or administering such antimicrobial composition. Examples include oral, parenteral (including subcutaneous, intradermal, intramuscular, intravenous and intraarticular), rectal and topical (including dermal, buccal, sublingual and intraocular) administration. An antimicrobial may be formulated as appropriate for such administration, which may be tailored to a given purpose, such as in a tablet, capsule, or other form for oral administration or injectable formulation for injection, or gel, cream, powder, ointment, rinse, lavage, or other composition for administration to a tissue layer, including rectal or dermal application. In some embodiments, one or more antimicrobial may be included in the surface of a material or an apparatus to be implanted on or within the body of a subject such as a human subject configured or otherwise formulated to have or promote an antimicrobial effect at the surface of such material or apparatus or to be released therefrom and have such an antimicrobial effect in tissue in the vicinity of such material or apparatus.

Antibiotics of any of a number of different classes or types may be used in accordance with the kits, systems, and methods disclosed herein. Examples include aminoglycosides, ansamycins, carbapenems, cephalosporins, antibiotic glycopeptides, lincosamides, antibiotic lipopeptides, macrolides, monobactams, nitrofurans, oxazolidinones, penicillins, quinolones, fluoroquinolones, sulfonamides, tetracyclines, or others. Any antibiotic from any of these categories may be used in accordance with aspects of the present disclosure. Non-limiting specific examples include, tobramycin, streptomycin, rifampicin, vancomycin, clindamycin, daptomycin, erythromycin, linezolid, penicillin, minocycline, pexiganan, fusidic acid, mupirocin, bacitracin, neomycin, polymixin B, and metronidazole. Other examples include metals or metal ions known to have antimicrobial or antibacterial effects, such as silver, copper, or zinc. In some examples, combinations of any two or more of the foregoing antibiotics or substances with antibiotic activity may be administered concurrently in accordance with an aspect of the present disclosure. In some examples, any one or more of the foregoing may also be explicitly excluded from use in accordance with an aspect of the present disclosure.

In one embodiment, the antimicrobial agent is an antibacterial, antifungal, antiviral, antiparasitic, antiseptic, bacteriostatic, bactericidal, or any combination thereof.

In one embodiment, the antimicrobial agent is selected from biguanide monomers and polymers, providone iodine, benzalkonium chloride, zinc chloride, sodium chlorite, silver, copper, palladium, gold, selenium, or any combination thereof. Chlorhexidine is a family member of biguanide monomers and polymers, along with other antiseptic compounds such as alexidine, polyaminopropyl biguanide (PAPB), and polyhexanide. The use of the antimicrobial implant surface components in combination with the other system components may allow tissue to better attach to the implant in an environment where infection by bacteria or other microorganisms is present or at high risk to become infected as compared to traditional implants. Improved tissue attachment in a high-risk infection environment may also provide better fixation of an implant. Titanium dioxide nanotube surfaces have been demonstrated to have increased vascularization of the implant surface relative to untreated titanium (Khosravi et al., “Nanosurfaces Modulate the Mechanism of Peri-Implant Endosseous Healing By Regulating Neovascular Morphogenesis,” Communications Biology 1:72 (2018), which is hereby incorporated by reference in its entirety). Improved vascularization and tissue attachment may also reduce bacterial attachment on the implant surface.

The kits, systems, and methods described herein may include two or more antimicrobial components, each component being directed to a different post-surgical tissue depth in a subject. The use of the components in combination (i.e., two or more) may reduce infections at various depths of a surgical wound or migration of bacteria from one depth of a surgical wound to another.

The kits, systems, and methods of the present disclosure may include at least two components selected from (i), (ii), (iii), and (iv). In one embodiment, the kit may include at least (i) and (ii). In another embodiment, the kit may include at least (i) and (iii). In yet another embodiment, the kit may include at least (i) and (iv). In yet another embodiment, the kit may include at least (ii) and (iii). In another embodiment, the kit may include at least (ii) and (iv). In another embodiment, the kit may include at least (iii) and (iv). In one embodiment, the kit includes two components selected from any combination of (i), (ii), (iii), and (iv).

Alternatively, the kits, systems, and methods of the present disclosure may include at least three components. In one embodiment, the kit may include at least comprises (ii) and (iv). In one embodiment, the kit may include at least (i), (ii), and (iii). In another embodiment, the kit may include at least (i), (ii), and (iv). In yet another embodiment, the kit may include at least (ii), (iii), and (iv). In yet another embodiment, the kit may include at least (i), (ii), (iii), and (iv). In one embodiment, the kit includes three components selected from any combination of (i), (ii), (iii), and (iv).

Alternatively, the kits, systems, and methods of the present disclosure may include four components selected from (i), (ii), (iii), and (iv).

In each of the various combinations available for the kits, systems, and methods described herein, a kit may include one or more of any component (i), one or more of any component (ii), one or more of any component (iii), and one or more of any component (iv).

The kit described herein may include one or more supplemental antimicrobial components. In one embodiment, the kit may include a supplemental antimicrobial component. Examples of supplemental antimicrobial components include any antimicrobial component described herein or known to those skilled in the art. In one embodiment, the supplemental antimicrobial component includes an antibiotic, an antimicrobial ion, a fungicide, an antiseptic, a nanoparticle, a nanostructure, a nanofiber, a polymer fiber, an electrospun fiber, an antimicrobial-containing particle, cellulose, a sugar, a ceramic fiber, a ceramic particle, a ceramic layer, an antibiotic-containing cement composition, or any combination thereof. A ceramic fiber, particle, or layer as described herein may include ablative and/or dissolvable surfaces (such as amorphous calcium phosphate, polyvinyl alcohol, rapidly biodegradable polymers, some sugars). In one example, a polymer fiber or particle may contain an antimicrobial agent. In one example, cement compositions may include one or more antibiotic, such as a parenteral, oral, and/or topical antibiotic composition, or any combination thereof. In one embodiment, the antimicrobial component may be supplied as a solution, gel, insert, coating, powder, spray, glue, adhesive, or delivered systemically. In one embodiment, the supplemental antimicrobial component may include, for example, a wound insert, a coating, surface composition, a gel, a solution, a glue, an adhesive, a barrier layer, a dressing, a suture, a staple, a wound closure device, a hydrogel, an antimicrobial ion, an antimicrobial protein, a detergent, a bactericidal solvent, an antiseptic, materials selected to create a galvanic reaction to induce a current, or a combination thereof.

The kits, systems, and methods described herein may include additional elements. For example, in one embodiment, the kit, system, and method may include an additional step, step (v), which includes instructions for sequential or simultaneous administration of at least two of (i), (ii), (iii), and (iv) to a patient.

The first component (i.e., the component directed to a first post-surgical tissue depth comprising a piece of surgical hardware) may include an antimicrobial surgical hardware.

When the first component includes antimicrobial surgical hardware, the antimicrobial surgical hardware may include any hardware used during a surgical procedure. In one embodiment, the surgical hardware is selected from the group consisting of an implant, an interbody cage, an interbody fusion, other spine implants, a cranial implant, a maxillofacial implant, trauma implant, a screw, a plate, a nail, a pedicle screw, an anchor, antibiotic eluting cement or polymer, antibiotic eluting cement or polymer spacer, a temporary spacer for revision surgery, a hip replacement device, a knee replacement device, a shoulder replacement device, a distal extremity implant, an intramedullary device, a wire, a pin, a staple, an external fixation device, a sports medicine implant, a dental implant, a pacemaker, a sleeve or pouch implanted with another device, a shunt, a hernia mesh, and any combination thereof.

As discussed supra, the first component may include antimicrobial surgical hardware and may be, for example, an implant with antimicrobial, bactericidal, anticolonization, anti-attachment, and/or bacteriostatic surface or attributes, or any combination thereof. An implant in accordance with the first component of the present disclosure includes, for example, one that has one or more surfaces resistant to bacterial attachment, or that include bactericidal agents, antiseptic agents, bactericidal or bacteriostatic drug agents, or any combination thereof. The first component may, for example, be designed to reduce the amount of live bacteria or reduce attachment, proliferation, or function of bacteria on an implant surface or in a surgical site for a period of time. The surface of a first component (e.g., an implant device) may be treated with at least one antimicrobial structure or compound and may be a combination of antimicrobial structures and/or antimicrobial drugs as described supra.

Examples of first components useful in accordance with the present disclosure include but are not limited to medical devices, silicon nanopoints, nanoPEEK, and graphene nanoflakes applied to surfaces. Examples of first components comprising an antimicrobial structure useful in accordance with the present disclosure further include calcium phosphate treated titanium nanotubes, nanotubes without calcium phosphate, nanotubes with calcium sulfate, nanotubes with nanocrystalline hydroxyapatite spikes, nanoblades, angular nanostructures, and rutile titanium nanotubes. Examples of first components comprising an antimicrobial agent or drug include but are not limited to silver, antiseptics, and antibiotics (used for example with hernia, pacemaker, and neural shunt meshes). Additional examples of first components comprising an antimicrobial agent or drug include but are not limited to titanium with silver, for example two-layer antimicrobial nanotubes (Ag and Ag+chlorhexidine), and one-layer antimicrobial nanotubes (Ag and Ag+chlorhexidine).

The second component (i.e., the component directed to a second post-surgical tissue depth comprising a deep tissue layer) may include an antimicrobial wound insert. Examples of an antimicrobial wound insert include but are not limited to an insert, mesh, bone cement, foam, gel, and a drainage device. Additional examples of an antimicrobial wound insert include, but are not limited to, a lavage, rinse, hemostat, and negative pressure wound therapy device. The second component may treat contaminated wounds and prevent the establishment of bacterial infections within a wound or prevent surface infections in tissues adjacent to a surgical implant or location of surgery, or tissue descending from the surface of a wound or surgical site. Examples of second components having an antimicrobial wound insert include drug specific surfaces, such as fully resorbable meshes, antibiotic loaded bone cements, antibiotic loaded adhesives, metal ion loaded hydroxyapatite, and resorbable internal sutures. Additional examples include administration of electric pulses with antibiotics, antibiotic loaded foam gelatin wound insert, chlorhexidine (for example, extended release chlorhexidine) in various forms such as a wash, lavage, hemostat, gel, negative pressure wound therapy device, wound insert, adhesive, and/or internal sutures. Examples of antimicrobial agents useful in the second component may include chlorhexidine, providone iodine, benzalkonium chloride, antibiotics or various other ingredients that reduce bacteria function or viability. For example, ZnCl₂, NaClO₂, or other bactericidal agents, antiseptic agents, or bacteriostatic agents may be useful as an antimicrobial agent of the second component.

The third component (i.e., the component directed to a third post-surgical tissue depth comprising a superficial tissue layer) may include an antimicrobial surgical site cleanser. Examples of an antimicrobial surgical site cleanser include but are not limited to an antimicrobial wash, a rinse, an irrigation, and a wound lavage. The third component is intended to treat contaminated wounds and prevent the establishment of bacterial infections within superficial tissue layer adjacent to one or more deep tissue layers after or during surgery (for example, implant surgery), and may prevent infections in tissue descending from the surface of a wound or surgical site. The third component may be used to clean a wound or surgical site and remove contaminating bacteria. Bacteria may be physically removed, as with a wash, or exposed to antimicrobial structures or drugs, such as antibiotics and/or nanoparticles. The components may act at the time of treatment with a short duration of effect or may have a more durable effect utilizing extended release drug systems or temporarily durable nanoparticles. Light and electric field systems may produce effects while they are in use. The third component may include, for example, one or more washes, irrigation, gel, negative pressure wound therapy device, adhesive, hemostat, or wound lavage with antimicrobial, bactericidal, anticolonization, anti-attachment, and/or bacteriostatic attributes. Examples of antimicrobial agents useful in the third component include biguanide monomers and polymers, such as chlorhexidine, alexidine, polyaminopropyl biguanide (PAPB), and polyhexanide, as well as providone iodine, benzalkonium chloride, antibiotics or various other ingredients that reduce bacteria function or viability. For example, ZnCl₂, NaClO₂, or other bactericidal agents, antiseptic agents, or bacteriostatic agents may be useful as an antimicrobial agent of the third component. The antimicrobial agent may be designed to reduce the amount of live bacteria in the internal tissue portion of a surgical wound or exposed hardware in a surgical site.

The fourth component (i.e., the component directed to a fourth post-surgical tissue depth comprising skin) may include an antimicrobial surgical dressing or a surgical site closure system or a surgical site protection system. Examples of an antimicrobial surgical dressing or a surgical site closure system or surgical site protection system include, but are not limited to, one or more bandages, wraps, wound dressings, wound coverings, negative pressure wound dressings, foam, sutures (e.g., external sutures), glue, adhesive, adhesive strips, a drainage tube, staples, a wound closure device, gauze, gel, ointment, a vacuum device, pad or dressing for use with a vacuum device, or any combination thereof. The antimicrobial surgical dressing or surgical site closure system or surgical site protection system may include antimicrobial, antiseptic, bactericidal, anticolonization, anti-attachment, and/or bacteriostatic agents or attributes, for example, by the administration of electric pulses or ultraviolet light.

Examples of wound closures and treatments that are suitable for use in the fourth component described herein include but are not limited to antibiotic loaded glue, antimicrobial sutures, antimicrobial bandages, antibiotic coverings, antibiotic gels, a vacuum device with or without washes, and light therapy. Useful light emitting therapy can emit any wavelength that provides a therapeutic effect. For example, the light therapy may be administered at between about 1 nm and 1,000 nm. In one embodiment, the light therapy is administered at a wavelength of between about 250 nm and about 500 nm. In one embodiment, the light therapy may be administered at about 300 nm, for example about 305 nm. Surgical dressing as described herein includes a protective dressing, an antibacterial dressing, an absorbent dressing, a debriding dressing, a skin adhesive dressing, and negative pressure wound therapy. Examples of surgical dressings include but are not limited to dry gauze, silicone, hydrogels, transparent films, impregnated gauze, antibacterial ointment, iodosorb, silver-based dressing, foam, collagen, alginates, hydrofibers, hydrocolloid, and any combination thereof.

The devices and materials used in any of the first component, second component, third component, and fourth component can be equipped with structures and compounds to prevent colonization of the device itself and inhibit a descending infection from forming.

As used herein, the term “simultaneous” therapeutic use refers to the administration of at least one additional agent beyond the two or more components for example, agents administered before, during, or after the administration of the two or more components, optionally, by the same route and at the same time or at substantially the same time. As used herein, the term “separate” therapeutic use refers to an administration of at least one additional agent beyond the two or more components, for example, agents administered before, during, or after administration of a two or more components, at the same time or at substantially the same time by different routes. As used herein, the term “sequential” therapeutic use refers to administration of at least one additional agent beyond the two or more components, for example, agents administered before, during, or after administration of the two or more components, at different times, the administration route being identical or different. More particularly, sequential use refers to the whole administration of the additional agent before administration of the two or more components. It is thus possible to administer the additional agent over several minutes, hours, or days before applying the two or more components. In one embodiment, the additional agent is administered before, during, or after the two or more components.

A second aspect of the present disclosure relates to a method of assembling the kit described herein.

This aspect is carried out in accordance with the previously described aspect.

A third aspect relates to an antimicrobial surgical system. The system comprises two or more antimicrobial components directed to two or more different post-surgical tissue depths in a subject for preventing, reducing, and/or treating surgical site infections.

This aspect is carried out in accordance with the previously described aspects.

A fourth aspect of the present disclosure relates to a method of assembling the system described herein.

This aspect is carried out in accordance with the previously described aspects.

A fifth aspect of the present disclosure relates to a method of preventing, reducing, and/or treating one or more surgical site infections. The method includes providing an antimicrobial surgical system including: two or more antimicrobial components directed to two or more different post-surgical tissue depths in a subject; and applying said antimicrobial surgical system to a subject under conditions effective to prevent, reduce, and/or treat one or more surgical site infections.

This aspect is carried out in accordance with the previously described aspects.

In one embodiment, the method includes providing (i) and (ii); or providing (i) and (iii); or providing (i) and (iv); or providing (ii) and (iii); or providing (ii) and (iv); or providing (iii) and (iv), in accordance with the previously described aspects. In another embodiment, the method includes providing (i), (ii), and (iii); or providing (i), (ii), and (iv); or providing (ii), (iii), and (iv), in accordance with the previously described aspects. In yet another embodiment, the method includes providing (i), (ii), (iii), and (iv), in accordance with the previously described aspects.

In one embodiment, the method includes administering a supplemental antimicrobial component, in accordance with the previously described aspects.

In one embodiment, the method includes a further step (v), for providing instructions for sequential or simultaneous administration of at least two of (i), (ii), (iii), and (iv) to a patient, in accordance with the previously described aspects.

In one embodiment, the method includes the step of selling by an individual or sales force or marketing, representing by a digital electronic means including a website or social media, representing on print media, representing on electronic media two or more of (i), (ii), (iii), and (iv) for use as a kit, system, together, as a product, or a bundle, or for a use comprising preventing, reducing, and/or treating one or more surgical site infections.

In one embodiment, the method includes the step of charging or receiving payment for two or more of (i), (ii), (iii), and (iv) for use as kit, system, together, as a product, or a bundle, or for a use comprising preventing, reducing, and/or treating one or more surgical site infections.

In one embodiment, the method includes the step of negotiating or executing a contract including two or more of (i), (ii), (iii), and (iv) to a customer for use as a kit, system, together, as a product, or a bundle, or for a use comprising preventing, reducing, and/or treating one or more surgical site infections, such negotiation or contract includes a combined price or for a single price for two or more of (i), (ii), (iii), and (iv). Such price may be lower or higher than the cost of the individual products.

In one embodiment, the first component is administered prior to administration of any of the second component, the third component, and the fourth component. In another embodiment, the second component is administered prior to administration of any of the first component, the third component, and the fourth component. In yet another embodiment, the third component is administered prior to administration of any of the first component, the second component, and the fourth component. In another embodiment, the fourth component is administered prior to administration of any of the first component, the second component, and the third component.

Any combination of two or more components may be included and expected to be useful in the kits, systems, bundles, methods, and devices disclosed herein. The kits, systems, bundles, methods, and devices disclosed herein may include multiple components, and may be used in combination drug therapy, or in combination with a drug device, or other combination with the intention of preventing, reducing, and/or treating infections or preventing, reducing, and/or treating surgical site infections when used together.

In the following description, reference is made to the accompanying drawings that form a part hereof, and in which is shown by way of illustration specific embodiments which may be practiced. These embodiments are described in detail to enable those skilled in the art to practice the disclosure, and it is to be understood that other embodiments may be utilized and that structural, logical and electrical changes may be made without departing from the scope of the present disclosure. The following description of example embodiments is, therefore, not to be taken in a limited sense.

The present disclosure may be further illustrated by reference to the following examples. The following examples are intended to illustrate, but by no means are intended to limit, the scope of the present disclosure as set forth in the appended claims.

### Example 1—Antimicrobial Nanotubes

NanoPEEK—PEEK was treated with ultrasonic impaction of various salts to create a nanostructured polymer surface. A variety of salts, ceramics, glasses, elements, or metals can be used in this process. Water soluble salts can easily be removed from the surface leaving a nanotextured PEEK surface. Non-water-soluble materials, such as hydroxyapatite, bioglass, titanium oxide or titanium particles, would be retained in the surface to provide a more biocompatible surface along with the nanotexturing of the PEEK provided in the process. A first treatment to create a nanotextured surface followed by the application of material to increase to biocompatibility or antimicrobial capacity of the surface can be added in a second process. The nanostructure increases the surface area and creates a texture that is smaller than the dimensions of bacteria. The increase in surface area was demonstrated to increase the attachment of eukaryotic cells, such as osteoblasts and stem cells. Literature has demonstrated dimensions in this range to inhibit/limit bacterial attachment.

Rutile titanium nanotubes—Work has been conducted on heat treating nanotubes to convert the titanium dioxide from an amorphous phase to either anatase or rutile crystalline forms of titanium dioxide. This heat treatment allowed the decrease in bacterial attachment relative to the amorphous form of titanium dioxide nanotubes. Titanium nanotubes are created by anodization in a fluoride containing electrolyte with available oxygen for oxidation of the titanium. Nanotubes are created with voltages between 5 and 30 V, typically in aqueous solutions. This process creates amorphous titanium oxide surfaces. The titanium bulk material is then heat treated to convert the amorphous titanium oxide to a rutile or anatase crystalline form. The altered structure and the atomic arrangement of the crystalline form limits the available charge groups on the surface. This reduces the ability of proteins and ions to bind to the surface and thus limits bacteria attachment to the surface.

Antimicrobial titanium nanotubes—First generation nanotubes (single layer) and second generation (two-layer), have been loaded with silver ions and silver ions with chlorhexidine. The silver ion elution allows the device to create a protective barrier on and around the implant surface. This helps to limit initial bacterial colonization and protect the surface from infections that occur for a period of days to weeks after surgery. The chlorhexidine provides a very short-term protection, on the order of hours. The chlorhexidine is immediately available upon contact with body fluids and the silver ions are available within hours after contact with body fluids. Extended release of chlorhexidine or other antimicrobials would be particularly useful for this system. Prolonging the antimicrobial release and antimicrobial effectiveness of the components is reasonably expected to reduce the number of live bacteria in or around the surgical site and specifically on the implant surface where it is common for a biofilm to form. Protecting implant surfaces from colonization is reasonably expected to reduce the infection rate for surgeries with permanently or temporarily implanted instrumentation.

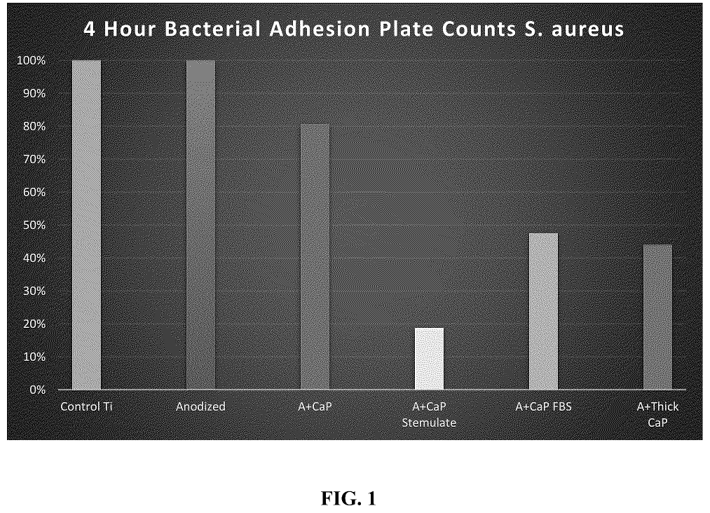

FIG. 1 shows four hour bacterial adhesion plate counts in S. aureus. The surface can reduce the number of bacteria that can actually attach to the surface from a physical mechanism demonstrated with the addition of amorphous calcium phosphate or the further adsorption of blood proteins to the nanosurface. The antimicrobial surface further has the ability to kill any bacteria that do become attached to the surface. This effectively reduces the number of viable bacterial cells that are needed to establish an infection on an implant.

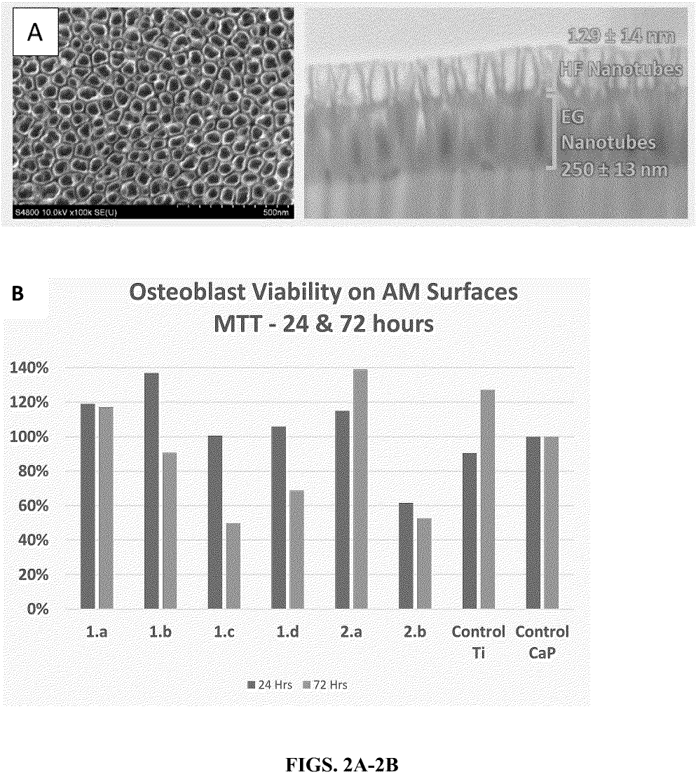

FIGS. 2A-2B show results of two-layer nanotubes (FIG. 2A) and 1-Layer AM surface—Cell viability (FIG. 2B). The biocompatibility of implants is measured in a direct contact assay by applying osteoblasts directly to the surface. Cells are quantified using a colorimetric assay to convert a substrate, MTT, to a formazan salt that has a brown color. Nanotubes were created by anodizing titanium in a hydrofluoric acid (HF) solution for a 1-layer surface or HF solution followed by a second anodization in ethylene glycol (EG) solution containing water and fluoride ions. Antimicrobial compounds are added to the created nanosurfaces and calcium phosphate is electrodeposited onto the surfaces.

### Example 2—Bacterial Inhibition Zones

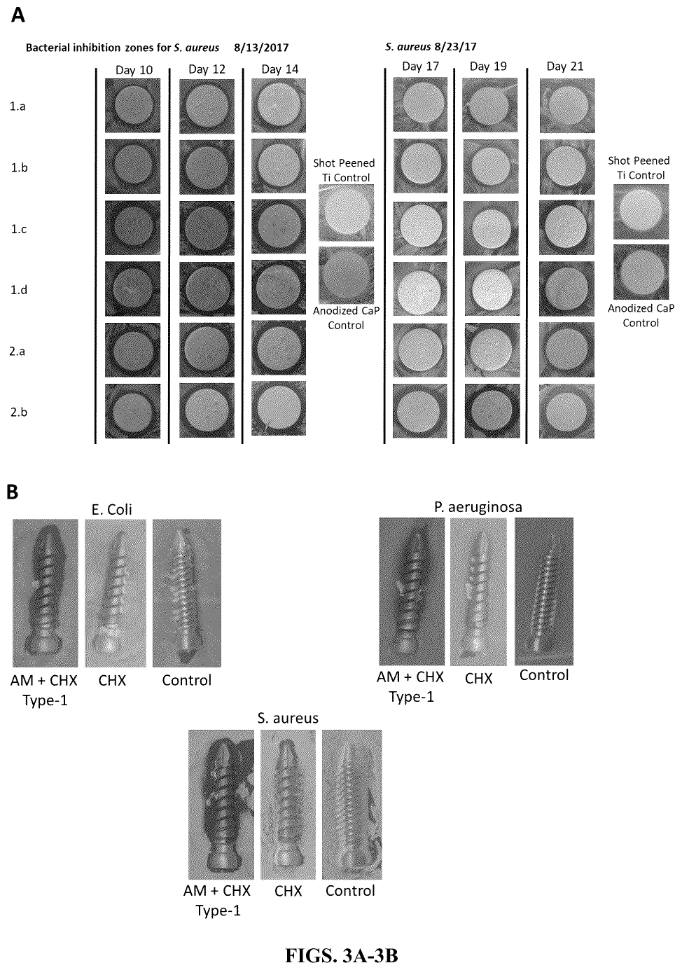

FIGS. 3A-3B show bacterial inhibition zones. FIG. 3A shows 1-Layer AM surface bacterial inhibition zones out to 21 Days. FIG. 3B shows 1-Layer AM surface with chlorhexidine bacterial inhibition zones on Day 1. Inhibition zones demonstrate the ability of the antimicrobial surfaces to limit bacterial growth immediately adjacent to the implant surface. The ability for the antimicrobial nanotube surface to function out to at least 21 days was demonstrated.

### Example 3—Improved Torque Value Using Bactericidal Pin

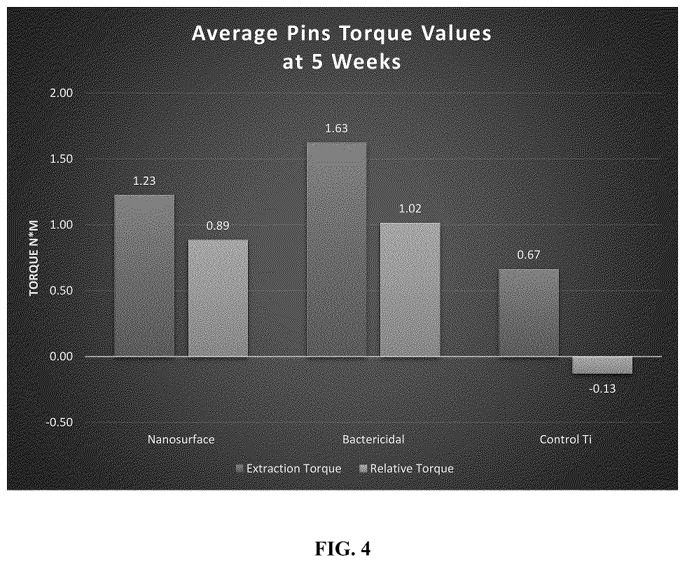

FIG. 4 shows extraction torque values from a pin external fixator study. Titanium ELI Grade 23 pins of 5 mm diameter were initially placed into the forelimb of a horse. Torque values were taken by measuring the peak torque that was required to rotate the pin 90 degrees. At the time of pin recovery from the forelimb at 5 weeks post-surgery, torque measurements were repeated in the same manner. Numerical values for each substrate were measured for total extraction torque and extraction torque minus the initial placement torque, giving a relative torque measurement. The nanosurface, with its ability to recruit vasculature to the surface and improve host tissue apposition was able to better retain initial fixation and improve upon that fixation when compared to control titanium, as measured by relative torque. The bactericidal surface was able to exceed the extraction and relative torque measurements of both the untreated titanium nanotube surface and the control titanium. This demonstrates the ability of both the titanium nanotube surface and the bactericidal titanium nanotube surface to increase the presence of eukaryotic cells at the implant and subsequently increase tissue integration to maintain and improve fixation in bone tissue in the face of descending infections.

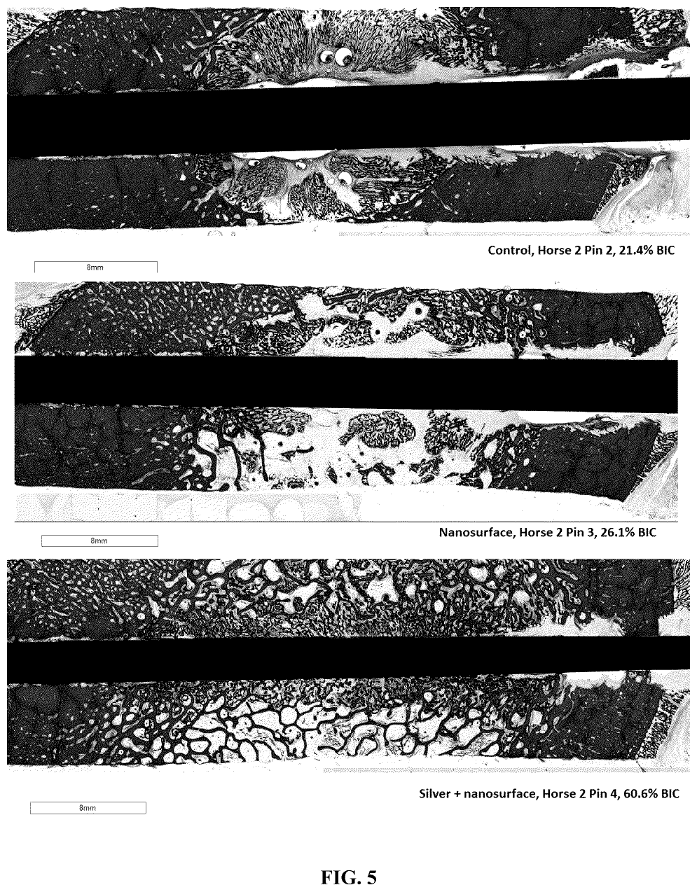

FIG. 5 shows horse pin histology after 5-week external fixator study. Increased bone implant contact on nanosurfaced and antimicrobial nanosurfaced pins. Pin samples were left in bone, fixed, cut into histological sections and stained with hematoxylin and eosin for histological analysis. Control pins, top image, show a portion of the pin in contact with bone where the dark blue bone material is adjacent to the black pin. A fibrous sheath of tissue is present surrounding the remainder of the pin. This is an area of bone loss due to infection that descended along the pin trac. The middle image demonstrates an unmodified nanosurface pin with some bone apposition and a small amount of fibrous tissue. This also demonstrates a descending infection along the pin trac but not as severe as the control pin. The bactericidal titanium nanosurface pin, bottom image, demonstrated much higher bone contact with the pin, relative to the other two groups. There is minimal sign of infection on the bactericidal pin. The 5-week timepoint exceeds the bactericidal lifetime of the pin but allowed the host tissue to win the race to the surface of the implant and resist a descending infection from loosening the pin.

### Example 4—Wound Insert

The insert is a lightly crosslinked foamed gelatin. The basis for the design is a gelatin foam hemostat. Antibiotics, rifampicin and minocycline, are combined with the gelatin to provide a burst release of the antibiotics when placed in the wound cavity. To further the effectiveness of the wound insert, resorbable polymer beads of PLGA loaded with antibiotics, rifampicin and minocycline, to extend the release of antibiotics to create a field of protection within the wound. Foams that have release their content of antibiotics but still remain in the wound can serve as a nidus for infections. The gelatin foam was designed to break down within a week. The discontinuous distribution of polymer microparticles would limit the spread of infections by forcing them to move through open tissue and the immune system rather than clinging to a continuous surface and creeping through the wound down to the implanted hardware. Early tests were conducted on gelatin foam inserts without polymer microparticles. As expected, the burst release was able to kill off all bacteria (10\hat{ }5 E. coli and 10\hat{ }5 S. aureus) in the first 24 hours. After 48 hours of elution, the foam did not have enough antibiotic remaining to kill bacteria. A rapid dissolving version of this wound insert was contemplated but never enabled. A bandage (for example, a band-aid) with the gelatin foam loaded with antibiotics could be used for wound closure to protect open wounds from becoming infected. The gelatin would break down within a few days making removal of the bandage and not disrupting granulation tissue a possibility.

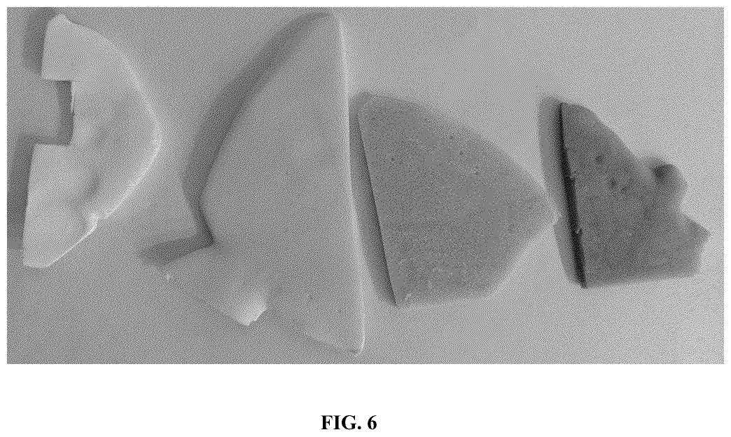

FIG. 6 shows gelatin foams with varying levels of Rifampicin and Minocycline antibiotics loaded into the gels prior to freezing and lyophilization. The structure of the inserts can be modified before freezing and lyophilization to achieve a specific structure or can be cut or machined after said processes. Rifampicin and minocycline were used as example drugs but any bacteria specific or broad-spectrum antibiotic or combination of antibiotics could be combined in this device.

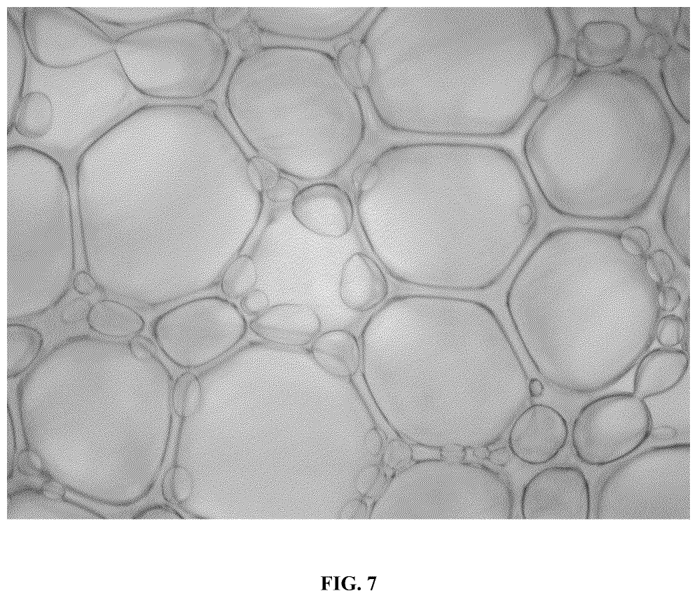

FIG. 7 shows a wound insert gelatin foam bubble structure after freezing and lyophilization to measure wall thickness. Bubble size and wall thickness helps to determine structural properties of the foam. The wall thickness also determines how much antibiotic material can be loaded into the structure. The lightly crosslinked nature of the gel allows it to be degraded in the body rapidly as to not provide a nidus for an infection.

The terminology used herein is for the purpose of describing particular embodiments only and is not intended to be limiting of the invention. As used herein, the singular forms “a”, “an” and “the” are intended to include the plural forms as well, unless the context clearly indicates otherwise. It will be further understood that the terms “comprise” (and any form of comprise, such as “comprises” and “comprising”), “have” (and any form of have, such as “has”, and “having”), “include” (and any form of include, such as “includes” and “including”), and “contain” (and any form of contain, such as “contains” and “containing”) are open-ended linking verbs. As a result, a method or device that “comprises,” “has,” “includes,” or “contains” one or more steps or elements possesses those one or more steps or elements, but is not limited to possessing only those one or more steps or elements. Likewise, a step of a method or an element of a device that “comprises,” “has,” “includes,” or “contains” one or more features possesses those one or more features, but is not limited to possessing only those one or more features. Furthermore, a device or structure that is configured in a certain way is configured in at least that way, but may also be configured in ways that are not listed.

Although preferred embodiments have been depicted and described in detail herein, it will be apparent to those skilled in the relevant art that various modifications, additions, substitutions, and the like can be made without departing from the spirit of the invention and these are therefore considered to be within the scope of the invention as defined in the claims which follow.

What is claimed is:

1. A kit for preventing, reducing, and/or treating one or more surgical site infections, the comprising:
two or more antimicrobial components directed to two or more different post-surgical tissue depths in a subject, wherein the two or more components are selected from: (i) a first component, the first component directed to a first post-surgical tissue depth comprising a surgical hardware; (ii) a second component, the second component directed to a second post-surgical tissue depth comprising a deep tissue layer; (iii) a third component, the third component directed to a third post-surgical tissue depth comprising a superficial tissue layer; and/or (iv) a fourth component, directed to a fourth post-surgical tissue depth comprising skin.

2. The kit of claim 1, wherein (i), (ii), (iii), and (iv) comprise at least one of an antimicrobial attachment surface and an antimicrobial agent.

3. The kit of claim 2, wherein the antimicrobial agent is an antibacterial, antifungal, antiviral, antiparasitic, antiseptic, bacteriostatic, bactericidal, or any combination thereof.

4. The kit of claim 2, wherein the antimicrobial agent is selected from the group consisting of biguanide monomers and polymers, providone iodine, benzalkonium chloride, zinc chloride, sodium chlorite, silver, copper, palladium, gold, selenium, or any combination thereof.

5. The kit of claim 1, wherein the kit comprises (i) and (ii).

6. The kit of claim 1, wherein the kit comprises (i) and (iii).

7. The kit of claim 1, wherein the kit comprises (i) and (iv).

8. The kit of claim 1, wherein the kit comprises (ii) and (iii).

9. The kit of claim 1, wherein the kit comprises (ii) and (iv).

10. The kit of claim 1, wherein the kit comprises (iii) and (iv).

11. The kit of claim 1, wherein the kit comprises (i), (ii), and (iii).

12. The kit of claim 1, wherein the kit comprises (i), (ii), and (iv).

13. The kit of claim 1, wherein the kit comprises (ii), (iii), and (iv).

14. The kit of claim 1, wherein the kit comprises (i), (ii), (iii), and (iv).

15. The kit of claim 1, wherein the kit comprises a supplemental antimicrobial component, wherein the supplemental antimicrobial component comprises at least one of an antibiotic, an antimicrobial ion, a fungicide, an antiseptic, a nanoparticle, a nanostructure, a nanofiber, a polymer fiber, a antimicrobial-containing particle, cellulose, a sugar, a ceramic fiber, a ceramic particle, a ceramic layer, an antibiotic-containing cement composition, or any combination thereof.

16. The kit of claim 1, the kit further comprising:
(v) instructions for sequential or simultaneous administration of at least two of (i), (ii), (iii), and (iv) to a patient.

17. The kit of claim 1, wherein the first component comprises an antimicrobial surgical hardware, wherein the antimicrobial surgical hardware is selected from the group consisting of an implant, an interbody cage, an interbody fusion, other spine implants, a cranial implant, a maxillofacial implant, trauma implant, a screw, a plate, a nail, a pedicle screw, an anchor, antibiotic eluting cement or polymer, antibiotic eluting cement or polymer spacer, a temporary spacer for revision surgery, a hip replacement device, a knee replacement device, a shoulder replacement device, a distal extremity implant, an intramedullary device, a wire, a pin, a staple, an external fixation device, a sports medicine implant, a dental implant, a pacemaker, a sleeve or pouch implanted with another device, a shunt, a hernia mesh, and any combination thereof.

18. The kit of claim 1, wherein the second component comprises an antimicrobial wound insert, wherein the antimicrobial wound insert is selected from the group consisting of an insert, mesh, bone cement, foam, gel, and drainage device.

19. The kit of claim 1, wherein the third component comprises an antimicrobial surgical site cleanser, wherein the antimicrobial surgical site cleanser is selected from the group consisting of an antimicrobial, an antimicrobial wash, a rinse, an irrigation, and a wound lavage.

20. The kit of claim 1, wherein the fourth component comprises an antimicrobial surgical dressing or surgical site closure system, wherein the antimicrobial surgical dressing or surgical site closure system is selected from the group consisting of a bandage, wrap, a wound dressing, a wound covering, a negative pressure wound dressing, foam, a suture, glue, adhesive, an adhesive strip, a drainage tube, a staple, a wound closure device, gauze, gel, ointment, a vacuum device, a pad or dressing for use with a vacuum device, and any combination thereof.

21. A method of preventing, reducing, and/or treating one or more surgical site infections, the method comprising:
providing an antimicrobial surgical system comprising:
two or more antimicrobial components directed to two or more different post-surgical tissue depths in a subject; and
applying said antimicrobial component to the subject under conditions effective to prevent, reduce, and/or treat one or more surgical site infections.

22. The method of claim 21, wherein the two or more components are selected from: (i) a first component, the first component directed to a first post-surgical tissue depth comprising a surgical hardware; (ii) a second component, the second component directed to a second post-surgical tissue depth comprising a deep tissue layer; (iii) a third component, the third component directed to a third post-surgical tissue depth comprising a superficial tissue layer; and/or (iv) a fourth component, directed to a fourth post-surgical tissue depth comprising skin.

23. The method of claim 22, wherein (i), (ii), (iii), and (iv) comprise at least one of an antimicrobial attachment surface and an antimicrobial agent, wherein the antimicrobial agent is an antibacterial, antifungal, antiviral, antiparasitic, antiseptic, bacteriostatic, bactericidal, or any combination thereof, and wherein the antimicrobial agent is selected from the group consisting of biguanide monomers and polymers, providone iodine, benzalkonium chloride, zinc chloride, sodium chlorite, silver, copper, palladium, gold, selenium, or any combination thereof.

24. The method of claim 22, wherein the method comprises:
providing (i) and (ii).

25. The method of claim 22, wherein the method comprises:
providing (i) and (iii).

26. The method of claim 22, wherein the method comprises:
providing (i) and (iv).

27. The method of claim 22, wherein the method comprises:
providing (ii) and (iii).

28. The method of claim 22, wherein the method comprises:
providing (ii) and (iv).

29. The method of claim 22, wherein the method comprises:
providing (iii) and (iv).

30. The method of claim 22, wherein the method comprises:
providing (i), (ii), and (iii).

31. The method of claim 22, wherein the method comprises:
providing (i), (ii), and (iv).

32. The method of claim 22, wherein the method comprises:
providing (ii), (iii), and (iv).

33. The method of claim 22, wherein the method comprises:
providing (i), (ii), (iii), and (iv).

34. The method of claim 21, the method further comprising:
administering a supplemental antimicrobial component, wherein the supplemental antimicrobial component comprises an antibiotic, an antimicrobial ion, a fungicide, an antiseptic, a nanoparticle, a nanostructure, a nanofiber, a polymer fiber, a antimicrobial-containing particle, cellulose, a sugar, a ceramic fiber, a ceramic particle, a ceramic layer, an antibiotic-containing cement composition, or any combination thereof.

35. The method of claim 22, the method further comprising:
(v) providing instructions for sequential or simultaneous administration of at least two of (i), (ii), (iii), and (iv) to a patient.

36. The method of claim 22, wherein the first component is administered prior to administration of any of the second component, the third component, and the fourth component.

37. The method of claim 22, wherein the second component is administered prior to administration of any of the first component, the third component, and the fourth component.

38. The method of claim 22, wherein the third component is administered prior to administration of any of the first component, the second component, and the fourth component.

39. The method of claim 22, wherein the fourth component is administered prior to administration of any of the first component, the second component, and the third component.

40. The method of claim 22, wherein the first component comprises an antimicrobial surgical hardware, wherein the antimicrobial surgical hardware is antimicrobial surgical hardware is selected from the group consisting of an implant, an interbody cage, an interbody fusion, other spine implants, a cranial implant, a maxillofacial implant, trauma implant, a screw, a plate, a nail, a pedicle screw, an anchor, antibiotic eluting cement or polymer, antibiotic eluting cement or polymer spacer, a temporary spacer for revision surgery, a hip replacement device, a knee replacement device, a shoulder replacement device, a distal extremity implant, an intramedullary device, a wire, a pin, a staple, an external fixation device, a sports medicine implant, a dental implant, a pacemaker, a sleeve or pouch implanted with another device, a shunt, a hernia mesh, and any combination thereof.

41. The method of claim 22, wherein the second component comprises an antimicrobial wound insert, wherein the antimicrobial wound insert is selected from the group consisting of an insert, mesh, bone cement, foam, gel, and drainage device.

42. The method of claim 22, wherein the third component comprises an antimicrobial surgical site cleanser, wherein the antimicrobial surgical site cleanser is selected from the group consisting of an antimicrobial, an antimicrobial wash, a rinse, an irrigation, and a wound lavage.

43. The method of claim 22, wherein the fourth component comprises an antimicrobial surgical dressing or surgical site closure system, wherein the antimicrobial surgical dressing or surgical site closure system is selected from the group consisting of a bandage, a wrap, a wound dressing, a wound covering, a negative pressure wound dressing, foam, suture, glue, adhesive, an adhesive strip, a drainage tube, a staple, a wound closure device, gauze, gel, ointment, a vacuum device, a pad or dressing for use with a vacuum device, and any combination thereof.

44. The method of assembling the kit of claim 1.
